(19) United States
(12) Patent Application Publication
(10) Pub. No.: US 2020/0248136 A1
(43) Pub. Date: Aug. 6, 2020

(54) BIOMIMETIC MICROTUBE AND PREPARATION METHOD THEREOF

(71) Applicant: National Tsing Hua University, Hsinchu City (TW)

(72) Inventors: Fan-Gang Tseng, Hsinchu City (TW); Long-Sheng Lu, Hsinchu City (TW); Hsueh-Yao Chu, Hsinchu City (TW); Ming-Hsuan Chiu, Hsinchu City (TW); Chun-Jieh Hsu, Hsinchu City (TW)

(73) Assignee: National Tsing Hua University, Hsinchu City (TW)

(21) Appl. No.: 16/395,268

(22) Filed: Apr. 26, 2019

(30) Foreign Application Priority Data
Feb. 1, 2019 (TW) 108103973

(51) Int. Cl. C12N 5/00; B29C 45/00; B29C 45/16

(52) U.S. Cl. CPC C12N 5/0075; B29C 45/0001; B29C 45/16; B29K 2505/00; C12N 2533/74; C12N 2535/00; B29K 2089/00; C12N 2533/54

(57) ABSTRACT

A biomimetic microtube and a preparation method thereof are provided. A coaxial pipe is used to form a biomimetic microtube having a core solution and a wall surrounding the core solution. In the preparation method, some various processing methods can be used to increase the roughness, porosity, and hardness of the wall of the biomimetic microtube.

### CROSS-REFERENCE TO RELATED APPLICATION

This application claims the priority benefit of Taiwan application serial no. 108103973, filed on Feb. 1, 2019. The entirety of the above-mentioned patent application is hereby incorporated by reference herein and made a part of this specification.

### BACKGROUND OF THE DISCLOSURE

### Field of the Disclosure

The disclosure relates to a hydrogel tube and a preparation method thereof, and specifically to a biomimetic hydrogel microtube and a preparation method thereof.

### Description of Related Art

Hydrogel is a biocompatible polymer with a three-dimensional network structure commonly used in tissue engineering. Hydrogel is cross-linked by monomers with hydrophilic functional groups, so it is highly absorbent but not dissolved in water. There is no standard for the water content in hydrogel. In general, gel containing 10-20 wt % or more of water is called hydrogel. Common hydrogels include collagen, gelatin, chitin, chitosan, alginate, cellulose and their hydrophilic derivatives, starch and hyaluronic acid, etc., all of which are biodegradable or bioabsorbable.

Since many important human tissues and organs have a fibrous or network structure, many studies have attempted to use these biocompatible hydrogel materials to prepare microfibers capable of carrying cells as biomimetic blood vessels.

### SUMMARY OF THE DISCLOSURE

The disclosure provides a preparation method of a biomimetic microtube. The preparation method includes providing a molding device having a coaxial pipe including an inner tube and an outer tube as well as a molding tank. The inner tube has an inner tube inlet and an inner tube outlet, and the outer tube has an outer tube inlet and an outer tube outlet. The outer tube sleeves outside the inner tube to form a coaxial pipe having two layers, and the outer tube outlet sleeves outside the inner tube outlet to form a coaxial outlet. The molding tank is used for receiving the coaxial outlet. Then, a molding liquid containing a divalent metal cation is injected into the molding tank so that the coaxial outlet is positioned below the liquid level of the molding liquid. Then, simultaneously, a gelatin solution is introduced from the inner tube inlet and an alginate solution with a monovalent metal cation is introduced from the outer tube inlet, and the two solutions are simultaneously introduced into the molding liquid from the coaxial outlet to form the biomimetic microtube. The biomimetic microtube has a core solution and a wall surrounding the core solution of the gelatin solution and the alginate solution.

According to some embodiments, the monovalent metal cation is sodium ion, potassium ion or a combination thereof, and the divalent metal cation is calcium ion, strontium ion or a combination thereof.

According to other embodiments, the alginate solution comprises microphase substances, and the microphase substances are a gas, a liquid, a solid or any combinations thereof.

According to still other embodiments, when the microphase substances are the gas, the liquid or a combination thereof, the biomimetic microtube is further heated to remove the microphase substances from the biomimetic microtube to form pores in the wall of the biomimetic microtube.

According to yet other embodiments, the inner surface of the outer tube, the outer surface of the inner tube, or both are etched to increase roughness thereof.

According to still other embodiments, the biomimetic microtube is further immersed in an aqueous solution of ethanol to dehydrate the biomimetic microtube.

The disclosure also provides a biomimetic microtube obtained by the above preparation methods, which can mimic the complex microenvironment of a specific cell and serve as a substitute for a biomimetic blood vessel.

The biomimetic microtube has a core solution and a wall surrounding the core solution. The core solution includes a core column from the gelatin solution as well as a mixed layer from a mixed solution of the gelatin solution and the alginate solution containing the monovalent metal cation. The wall is from the alginate cross-linked by the divalent metal cation.

According to some embodiments, the microphase substances are distributed in the wall.

According to other embodiments, the wall has pores left after the removal of the microphase substances.

According to still other embodiments, the wall has projections caused by the microphase substances or has stripe patterns left after etching the inner tube, the outer tube, or both.

The disclosure further provides a connecting structure for a biomimetic microtube and a needle tube. The connecting structure comprises a biomimetic microtube, a needle tube sleeved in the biomimetic microtube, and a heated heat-shrinkable tube sleeving outside the overlapping segment of the needle tube and the biomimetic microtube.

According to still other embodiments, the outer diameter of the needle tube and the inner diameter of the biomimetic microtube differ by less than 0.2 mm.

Based on the above, the wall of the biomimetic microtube contains an alginate cross-linked by a divalent metal cation, so that it not only has the advantages of biocompatibility and non-toxicity, but also has sufficient pressure resistance and good permeability. Moreover, the roughness, porosity and hardness of biomimetic microtube can be changed by various methods to simulate the condition of various blood vessels.

In order to make the aforementioned features and advantages of the disclosure more comprehensible, embodiments accompanying figures are described in detail below.

### BRIEF DESCRIPTION OF THE DRAWINGS

FIG. 1 is a schematic view of a molding device for a biomimetic microtube and a cross-sectional structure of an obtained biomimetic microtube according to some embodiments of the disclosure.

FIGS. 2A-2B are images of biomimetic microtubes respectively observed under an optical microscope and obtained through a molding experiment of biomimetic microtube in Examples 1-2.

FIG. 3A shows the original shape of fibroblast.

FIGS. 3B-3C show the shapes of fibroblasts at different cultivation times in Examples 3-4, respectively.

FIG. 4 shows a SEM image of a wall of a biomimetic microtube prepared by using an etched needle tube.

FIG. 5 shows compared statistical results of the cell adhesion experiments performed by using biomimetic microtubes respectively prepared through unetched and etched needle tubes.

FIG. 6A is a SEM image of a biomimetic microtube without adding any microphase substances.

FIG. 6B is a SEM image of a biomimetic microtube with micro-oil droplets added to an alginate solution.

FIG. 6C is an image of a biomimetic microtube under an optical microscope after addition of microbubbles.

FIG. 7A shows a SEM image of a calcium alginate pillar obtained after dehydration by using 75 vol % ethanol.

FIG. 7B shows a SEM image of a calcium alginate pillar obtained after dehydration by using 95 vol % ethanol.

FIG. 8 shows compression test results of calcium alginate pillars by using a tensile testing machine, wherein the vertical axis represents the compressing force applied by the machine, and the horizontal axis represents the compression distance.

FIG. 9 shows the statistical results of cell adhesion experiments after dehydration of the biomimetic microtubes with different concentrations of ethanol, wherein the number at each numerical point represents the number of attaching cells.

FIG. 10 shows a measurement device for measuring the maximum withstand pressure of a biomimetic microtube.

### DESCRIPTION OF EMBODIMENTS

The cross-linked hydrogel has a sponge-like porous structure, so the porosity and size of the pores in the cross-linked hydrogel can be controlled through the concentrations and types of the hydrogel and cross-linking agent, thereby regulating the permeability and mechanical strength of the obtained hydrogel. Therefore, if a biocompatible cross-linked hydrogel is used as the wall of the microtube, it is possible to simulate the strength and permeability of the wall of a real blood vessel.

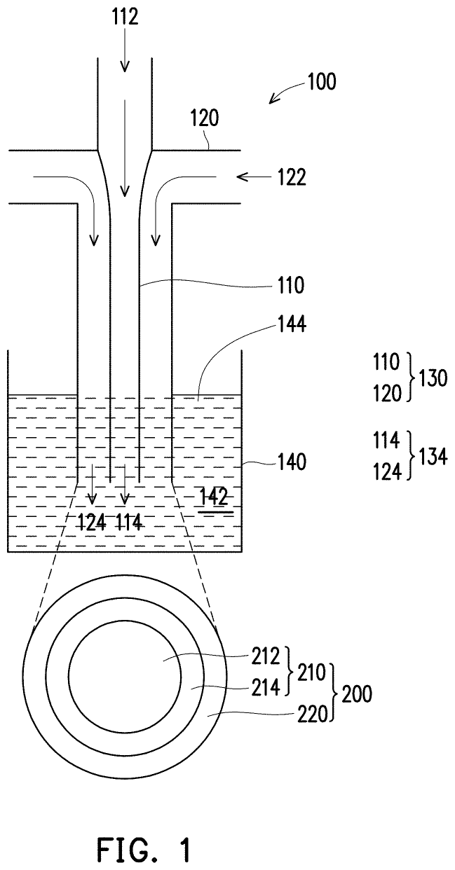

Please refer to FIG. 1, which is a schematic view of a molding device for a biomimetic microtube and a cross-sectional structure of an obtained biomimetic microtube according to some embodiments of the disclosure. In FIG. 1, a molding device 100 for preparing a biomimetic microtube has an inner tube 110, an outer tube 120, and a molding tank 140. The inner tube 110 has an inner tube inlet 112 and an inner tube outlet 114. The outer tube 120 has an outer tube inlet 122 and an outer tube outlet 124. The outer tube 120 sleeves outside the inner tube 110 to form a coaxial pipe 130. The outer tube outlet 124 sleeves outside the inner tube outlet 114 to form a coaxial outlet 134. The molding tank 140 is used for receiving the coaxial outlet 134.

Then, a biomimetic microtube is prepared by using the molding device of biomimetic microtubes, and the preparation method is described below. A molding liquid 142 containing a divalent metal cation is injected into the molding tank 140, and the coaxial outlet 134 is positioned below the liquid surface 144 of the molding liquid 142. Next, a gelatin solution is introduced from the inner tube inlet 112 and an alginate solution containing a monovalent metal cation is introduced from the outer tube inlet 122. Moreover, the two solutions are simultaneously introduced into the molding liquid 142 from the coaxial outlet 134, and the monovalent metal cation in the alginate solution and the divalent metal cation in the molding liquid 142 are ion-exchanged to crosslink the alginate, thereby forming a wall 220 of the biomimetic microtube 200.

Within the wall 220 of the biomimetic microtube 200 is a core solution 210 having a core column 212 and a mixed layer 214, wherein the core column 212 is made from a gelatin solution, and the mixed layer 214 is substantially made from a mixed solution of the gelatin solution and the non-cross-linked alginate solution. The wall 220 is substantially made from the alginate cross-linked by the divalent metal cations.

According to some embodiments, the monovalent metal cation in the alginate solution introduced into the outer tube inlet 122 may be, for example, sodium ion, potassium ion, or a combination thereof.

According to still other embodiments, the divalent metal cation in the molding liquid 142 is a cross-linking agent for alginate, which may be, for example, calcium ion, strontium ion, or a combination thereof. The anion in the molding liquid 142 may be, for example, chloride ion. The concentration of the divalent metal cation in the molding liquid 142 may be, for example, at least 0.1 M, so that the alginate can be rapidly cross-linked to form insoluble gel.

According to other embodiments, the concentration of the gelatin solution introduced from the inner tube inlet 112 can be, for example, 4-8.5 wt %, so that the gelatin solution has a suitable viscosity to allow the cells to attach to the wall 220 and grow easily. The concentration of the alginate solution containing the monovalent metal cation introduced from the outer tube inlet 122 may be, for example, 2-10 wt %, such that the alginate solution has a suitable concentration to facilitate formation of a biomimetic tube of appropriate hardness.

According to yet other embodiments, in the molding device of biomimetic microtube, some combinations of the inner tube 110 and the outer tube 120 having different tube diameters may be used to change the inner diameter, outer diameter and thickness of the biomimetic microtube. There is no specific limitations to the diameter of the inner tube 110 and the outer tube 120 as long as the microtube can be molded and meet the requirement. For example, when simulating microvessels, since the wall thickness of the microvessels is 0.1 mm at maximum, the difference between the inner diameter of the outer tube 120 and the outer diameter of the inner tube 110 may be 0.3 mm, and then the flow rate of gelatin solution in the inner tube 110 can be controlled to prepare a biomimetic microtube having a wall thickness of about 0.1 mm.

According to some embodiments, the core solution 210 in the biomimetic microtube 200 can be washed away with water to obtain a hollow biomimetic microtube which has the wall 220 composed of the cross-linked alginate only.

According to still other embodiments, the outer surface of the inner tube 110, the inner surface of the outer tube 120, or both may be etched to increase the roughness thereof. Therefore, the surface of the wall 220 of the obtained biomimetic microtube 200 may have stripe pattern disposed thereon to increase the roughness of the surface of the wall 220.

According to still other embodiments, a plurality of microphase substances may be added to the alginate solution. The microphase substances may be a gas, a liquid, a solid, or any combinations thereof and have a size of about micrometer. For example, the gas may be bubbles, the liquid may be oil droplets, and the solid may be particles. This method allows the surface of the wall of the biomimetic microtube to have granular projections, which increases the wall roughness of the obtained biomimetic microtube. The material of the microphase substances may be selected from any materials that are not dissolved in the alginate solution containing monovalent metal cations and the molding liquid. In addition, when the microphase substance is a gas or a liquid, the biomimetic microtube may be further heated to allow the gaseous or liquid microphase substance to leave the biomimetic microtube, thereby forming pores in the wall of the biomimetic microtube.

According to still other embodiments, the biomimetic microtube may be further immersed in an aqueous solution of ethanol with different concentrations to dehydrate the biomimetic microtube and increase the hardness of the wall of the biomimetic microtube.

Next, coaxial needle tubes having two needle tubes with different diameters were used as the coaxial pipe 130 in the following experiments.

### Embodiment 1: Molding Experiment of Biomimetic Microtube

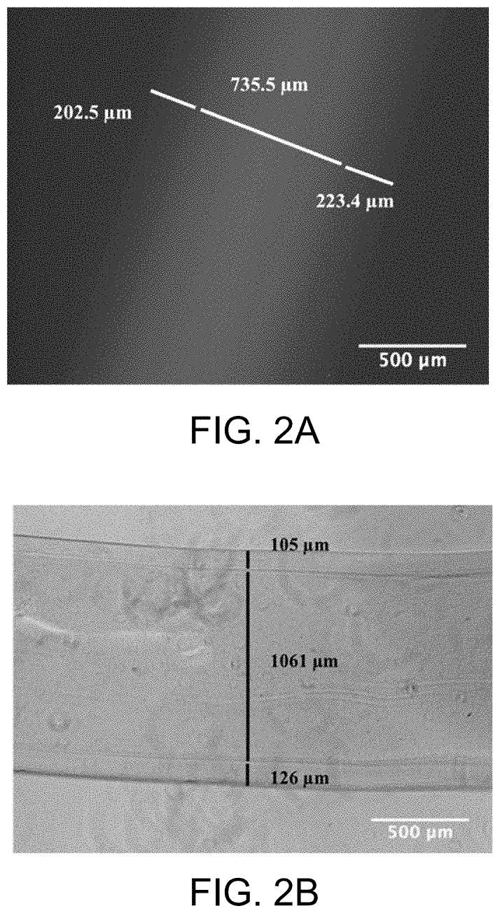

In this embodiment, a molding experiment of a biomimetic microtube was carried out. See Table 1 for relevant experimental parameters. Please refer to Table 1 and FIGS. 2A-2B for the experiment results. As can be seen from Table 1 and FIGS. 2A-2B, when the outer needle tubes of the coaxial needle tubes are selected to have the same size, the smaller the size of the inner needle tube was, the thicker the wall thickness of the obtained biomimetic microtube was.

TABLE 1
Experimental data and results of biomimetic
microtube molding experiments

| Examples | | 1 | 2 |
|---|---|---|---|
| Number, inner diameter/outer | | No. 20 | No. 19 |
| diameter (mm) of inner needle tube | | 0.60/0.90 | 0.77/1.07 |
| Number, inner diameter/outer | | No. 16 | No. 16 |
| diameter (mm) of outer needle tube | | 1.37/1.67 | 1.37/1.67 |
| Inner needle | concentration (wt %) | 5 | 5 |
| tube: | flow rate (ml/h) | 4 | 20 |
| gelatin solution | | | |
| Between inner | concentration (wt %) | 4 | 2 |
| and outer | flow rate (ml/h) | 9 | 12 |
| needle tubes: | | | |
| alginate | | | |
| solution | | | |
| Molding liquid: SrCl₂ solution (M) | | 0.1 | 0.1 |
| Biomimetic | Core column (μm): | ~700-900 | ~1000 |
| microtube | gelatin solution | | |
| | Thickness of mixed | | |
| | layer (pm): | | |
| | Mixed solution of | | |
| | alginate/gelatin | | |
| | Thickness of wall (μm): | ~200-400 | ~100 |
| | Cross-linked alginate | | |

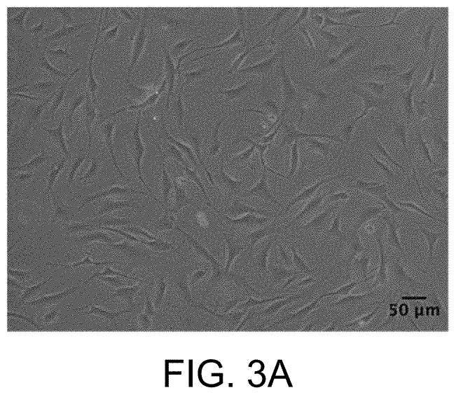

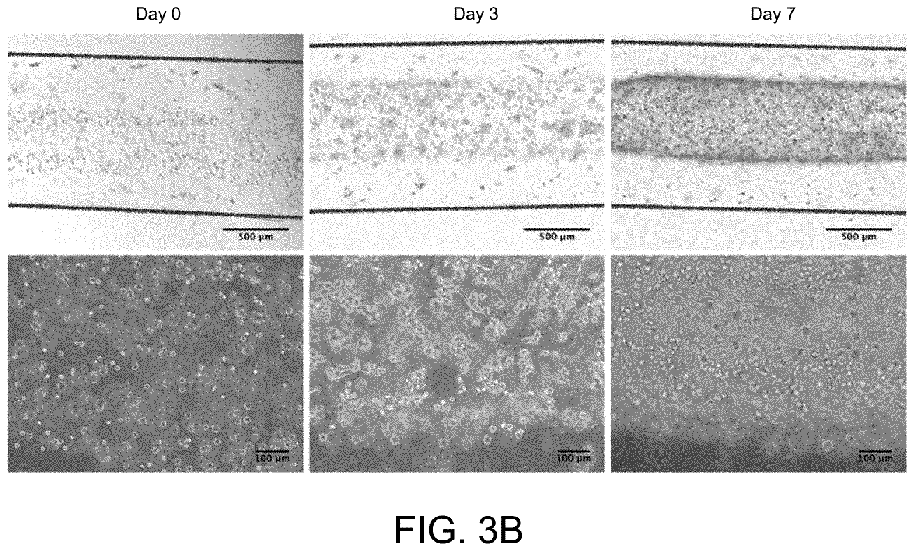

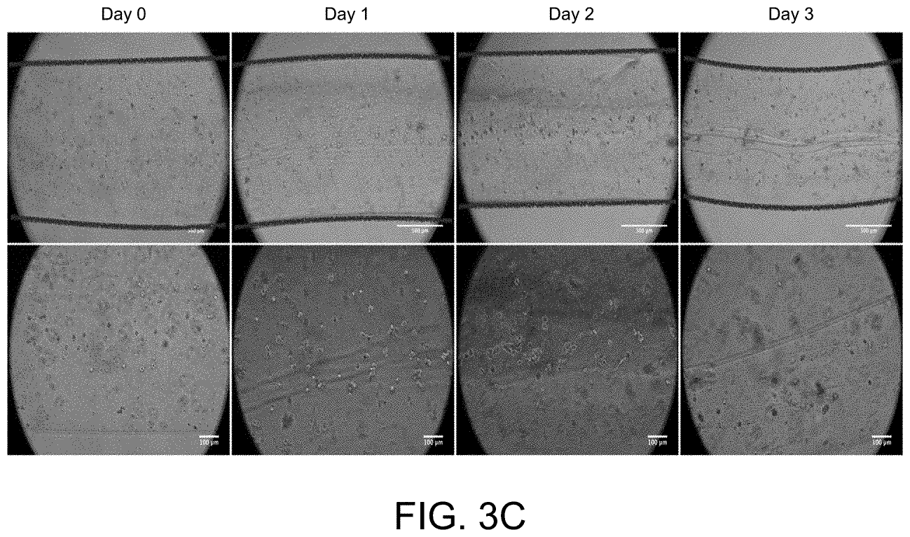

Next, cell adhesion experiments were performed on the biomimetic microtubes. In the gelatin solution that is introduced into the inner tube, fibroblasts were added first. After the biomimetic microtubes were molded, it was tested whether the fibroblasts can grow smoothly in the biomimetic microtubes. The original shape of the fibroblasts is shown in FIG. 3A. See Table 2 for the relevant experimental parameters, see FIG. 3B for the results obtained in Example 3, and see FIG. 3C for the results of Example 4.

TABLE 2
Experimental data and results of culture of fibroblasts in
biomimetic microtubes.

| Examples | | 3 | 4 |
|---|---|---|---|
| Number, inner diameter/outer diameter | | No. 19 | No. 19 |
| (mm) of inner needle tube | | 0.77/1.07 | 0.77/1.07 |
| Number, inner diameter/outer diameter | | No. 16 | No. 16 |
| (mm) of outer needle tube | | 1.37/1.67 | 1.37/1.67 |
| Inner needle tube: gelatin solution + | | 5 | 5 |
| fibroblasts* (wt %) | | | |
| Between inner and outer needle tubes: | | 4 | 2 |
| alginate solution (wt %) | | | |
| Molding liquid: SrCl₂ solution (M) | | 0.1 | 0.1 |
| Biomimetic | Outer tube | 1370 | 1370 |
| microtube | diameter (μm) | | |
| | Wall thickness (μm) | 300 | 100 |
| cell culture | FBS** (wt %) | 10 | 15 |
| soliution | | | |

* The culture temperature was 37° C.
**fetal bovine serum (FBS)

In Example 3, as can be seen from FIG. 3B, on day 0, the cells in the biomimetic microtubes were separated one by one, looked like the cells just cut in FIG. 3A, and some cells still flowed out from the front and rear ends of the biomimetic microtubes. On day 3, the positions of the cells were almost fixed and the cells began to grow. On day 7, the cells almost filled up the space in the biomimetic microtubes.

In Example 4, the number of cells added to the gelatin solution was small. As can be seen from FIG. 3C, on day 0, the cells in the hydrogel tube were in round shape, and the cells began to stretch after the first day. Two days later, more and more cells expanded outward from the center point, indicating that the cells had begun to split and grow. Therefore, it can be known that concentration of hydrogel, hydrogel wall and concentration of serum have a direct influence on biological behaviors, such as cell proliferation, migration and adhesion.

Comparing Examples 3-4, it is known that when the wall thickness of the biomimetic microtube is reduced, the cell culture medium outside the biomimetic microtube can enter the biomimetic microtube more easily, and the increased concentration of FBS in the cell culture medium can effectively improve the growth rate of the cells.

### Embodiment 2: Etching the Surface of the Needle Tube—Changing the Roughness of the Surface of Biomimetic Microtube

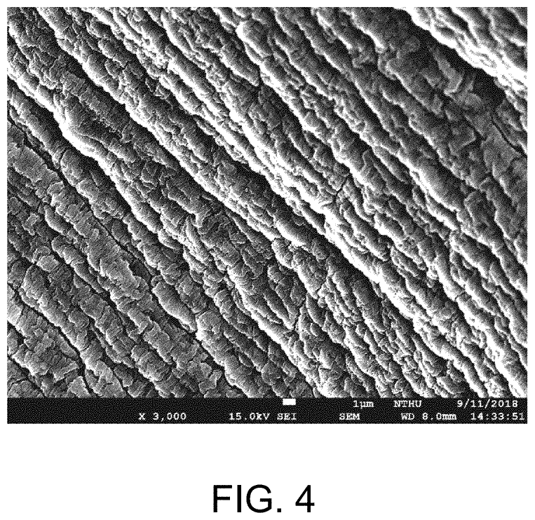

In this embodiment, etching is used to increase the roughness of the inner surface of the outer needle tube, the outer surface of the inner needle tube or both. Therefore, the roughness of the outer surface, the inner surface or both of the walls of the biomimetic microtubes may be increased separately to test whether the wall roughness of the biomimetic microtube affects the adhesion of the cells. The outer surface of the inner needle tube may be etched mechanically, for example, by using sandpaper to rub the outer surface of the inner needle tube to create scratches on the outer surface of the inner needle tube. The inner surface of the outer needle tube may be etched by using a chemical etchant to increase the roughness of the inner surface of the outer needle tube. FIG. 4 shows a SEM image of a wall of a biomimetic microtube prepared by using an etched needle tube. As can be seen from FIG. 4, there are a plurality of stripe scratches on the wall of the biomimetic microtube.

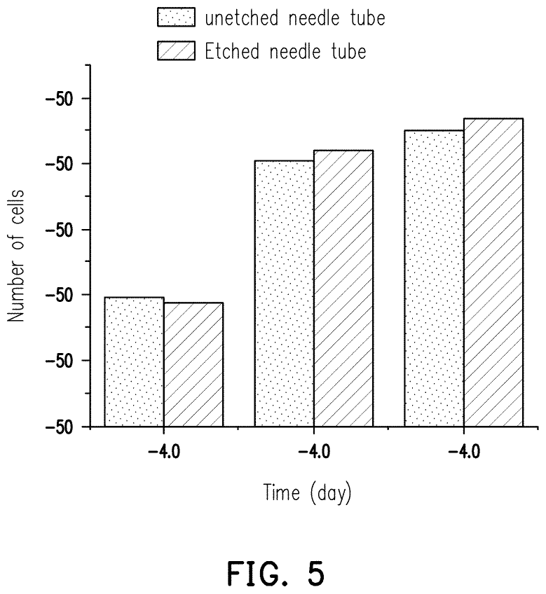

Next, an experiment in which cells attaching to the biomimetic microtube was carried out. In this experiment, the MCF-7 cell clone of the breast cancer cells was added to the cell culture medium, and the prepared biomimetic microtube injected with the cell culture medium was immersed in a cell culture solution containing the MCF-7 cell clone for a period of time. Observation was carried out continuously to observe the attachment of cells to the biomimetic microtubes. FIG. 5 shows compared statistical results of the cell attachment experiments performed by using biomimetic microtubes respectively prepared through unetched and etched needle tubes. It can be seen from FIG. 5 that there is no significant difference in the number of cells attaching to the biomimetic microtubes prepared by using the unetched and etched needle tubes. The number of cells attaching to the biomimetic microtubes prepared by using the etched needle tube is slightly larger. The roughness of the surface of needle tubes should be further adjusted to find the optimized size of scratch.

### Embodiment 3: Adding Microphase Substance—Changing the Roughness and Porosity of the Surface of Biomimetic Microtubes

In this embodiment, the roughness of the wall surface of the biomimetic microtubes is increased by adding a microphase substance to the alginate solution. The microphase substance is not particularly limited as long as it is not dissolved in the alginate solution and the molding liquid. For example, the microphase substance may be a gas, a liquid, a solid, or any combinations thereof, such as bubbles, oil droplets or microparticles, etc., which allows the wall surface of the biomimetic microtubes to have granular projections. Therefore, when the biomimetic microtubes were molded, the microphase substance is distributed in the wall of the biomimetic microtubes and the mixed layer along with the alginate solution.

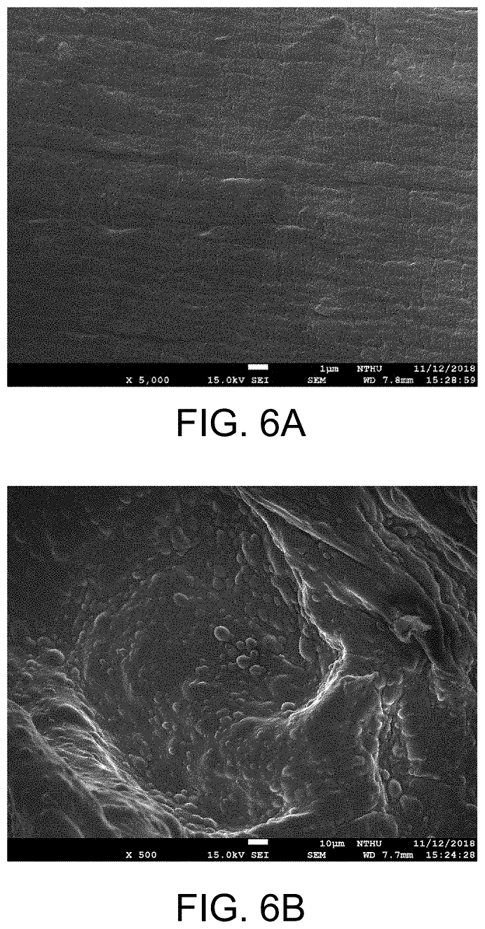

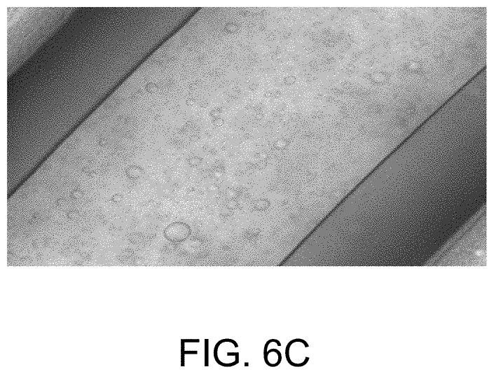

The obtained results are shown in FIGS. 6A-6C, wherein FIG. 6A is a SEM image of a biomimetic microtube without the addition of any microphase substances, FIG. 6B is a SEM image of a biomimetic microtube with micro-oil droplets added to the alginate solution, and FIG. 6C is an image of a biomimetic microtube under an optical microscope after addition of microbubbles. It can be seen that the wall surface of the biomimetic microtube in FIG. 6A is smooth, and the walls of the biomimetic microtube in FIGS. 6B-6C have granular projections.

In addition, when the microphase substance is gas, liquid or both, the biomimetic microtube may be further heated to allow the gas to escape or the liquid to flow out from the wall of the biomimetic microtube to form pores in the wall of the biomimetic microtube, and the porosity of the wall of biomimetic microtube is thus increased.

### Embodiment 4: Dehydration—Changing the Wall Hardness of Biomimetic Microtubes

In this embodiment, the ethanol dehydration method is used to increase the wall hardness of the biomimetic microtubes. In general, the wall hardness of the biomimetic microtube can be increased by only increasing the concentration of alginate in the alginate solution. However, increasing the concentration of alginate in the solution tends to make the viscosity of the alginate solution become too high, and the difficulty in preparing the biomimetic microtubes is thus increased. Therefore, immersing the biomimetic microtubes in ethanol to dehydrate the biomimetic microtubes can also increase the wall hardness of the biomimetic microtubes.

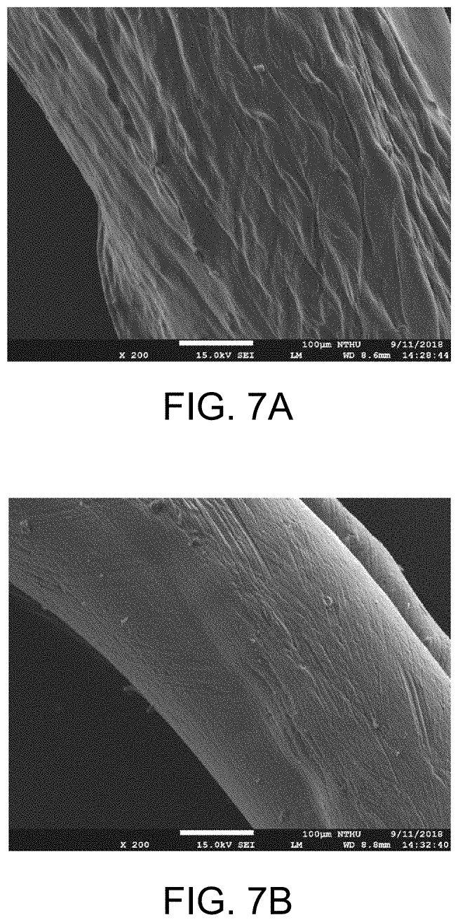

Here, in order to facilitate the hardness test, the sample to be tested is a solid column of calcium alginate having a diameter of 5 mm. FIG. 7A shows a SEM image of a biomimetic microcolumn obtained after dehydration by using an ethanol aqueous solution with a concentration of 75 vol %, which shows that the surface of the dehydrated calcium alginate pillar is slightly wrinkled due to dehydration. FIG. 7B shows a SEM image of a dehydrated biomimetic microcolumn obtained after dehydration by using an ethanol aqueous solution with a concentration of 95 vol %, which shows that the surface of the calcium alginate pillar subjected to a higher degree of dehydration is relatively smooth.

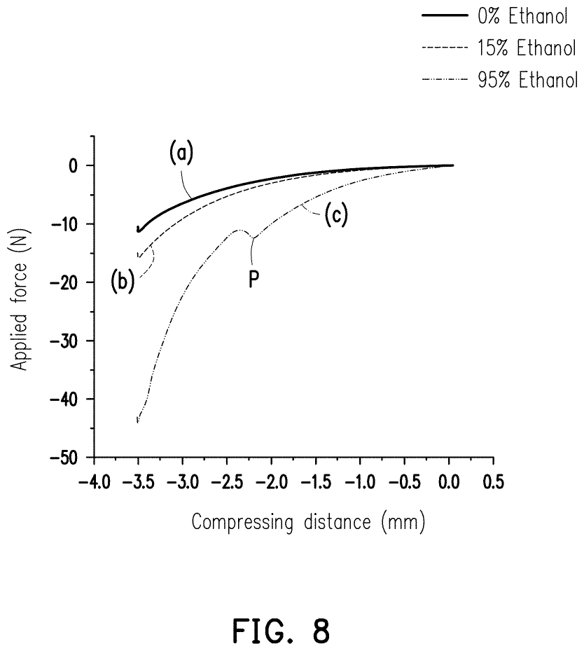

FIG. 8 shows the compression test results of calcium alginate pillars by using a tensile testing machine. The vertical axis represents the compressing force applied by the machine, and the horizontal axis represents the compressing distance. In FIG. 8, the curve (a) is obtained from the undehydrated biomimetic microcolumn, the curve (b) is obtained from the biomimetic microcolumn dehydrated by using an aqueous ethanol solution with a concentration of 15 vol %, and the curve (c) is obtained from the biomimetic microcolumn dehydrated by using an aqueous ethanol solution with a concentration of 95 vol %. It can be seen from FIG. 8 that when the biomimetic microcolumns are subjected to a higher degree of dehydration, a greater external force is required to the same compressing distance, which indicates that the hardness of the biomimetic micro subjected to a higher degree of dehydration is greater. Specifically, for the biomimetic microcolumn dehydrated by using the 95 vol % ethanol solution, when the compressing force is about 15 N, the curve (c) has a discontinuous point P, indicating that there is a phenomenon of fracture.

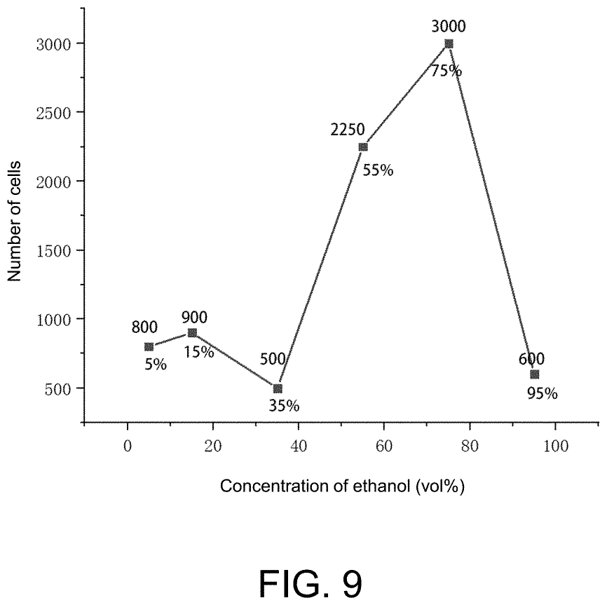

FIG. 9 shows the statistical results of cell attachment experiments after dehydration of the biomimetic microtubes with different concentrations of ethanol, wherein the number at each numerical point represents the number of the attaching cells. The cell attachment experiment performed in this embodiment is the same as the cell attachment experiment performed in the Embodiment 2, and thus the details thereof will not be described again. As can be seen from FIG. 9, the number of cells attaching to the biomimetic microtubes dehydrated by using 75 vol % ethanol solution was the highest, indicating that it brings the optimal cell adhesion effect.

### Embodiment 5: Maximum Tolerable Pressure of Biomimetic Microtubes

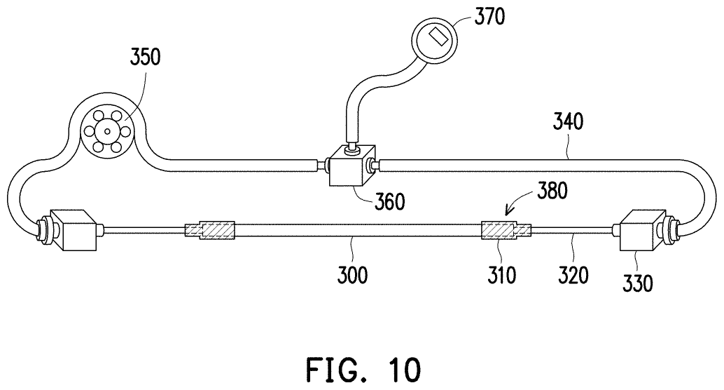

The measuring device used in this embodiment is shown in FIG. 10. In FIG. 10, a biomimetic microtube 300 having a larger diameter sleeves outside a needle tube 320 having a smaller diameter to form an overlapping connecting segment 380. Thereafter, a heat-shrinkable pipe 310 sleeves outside the overlapping connecting segment 380. At present, it has been found in the experiment that when the difference between the inner diameter of the biomimetic microtube 300 and the outer diameter of the needle tube 320 is less than 0.2 mm, it may effectively prevent leakage from the connecting segment 380.

Then, the needle tube 320 is connected to a hose 340 by a joint 330, and the hose 340 is connected to a peristatic pump 350 to change the pressure applied to the liquid in the pipeline. In addition, a pressure gauge 370 may be externally connected to the hose 340 by using a three-way valve 360 to measure the pressure of the liquid in the pipeline.

The biomimetic microtubes 300 under test were prepared by using solutions with different concentrations of sodium alginate. During the test, a dyed water was introduced into the pipeline. The flow rate of the liquid in the pipeline is controlled by the rotational speed of the peristatic pump 350. The greater the rotational speed of the peristatic pump 350 was, the greater the flow rate of the liquid in the pipeline was, and the higher the displayed liquid pressure in the tube was. The maximum tolerable pressure of the obtained biomimetic microtubes 300 are shown in Table 3. The pressure tolerable range of different blood vessels in the human body is shown in Table 4.

Comparing the results of Table 3 with the values of Table 4, it can be seen that the biomimetic microtube with a wall thickness of only 100-200 μm in Example 5 was able to withstand the pressure range of the artery, and therefore the biomimetic microtubes may serve as simulation models of blood vessels. In addition, the rotational speed of the peristatic pump in Examples 7-9 had reached a maximum value, and therefore the maximum tolerable pressure of the biomimetic microtubes of Examples 7-9 should be greater than the values listed in Table 3.

TABLE 3
Maximum withstand pressure test of biomimetic microtubes

| Example | 5 | 6 | 7 | 8 | 9 |
|---|---|---|---|---|---|
| Concentration (wt %) | 2 | 2 | 3 | 4 | 6 |
| of alginate solution | | | | | |
| wall thickness (μm) of | 100-200 | 200-400 | 200-400 | 200-400 | 200-400 |
| biomimetic microtube | | | | | |
| pressure (mmHg) of | 40.85 | 387.86 | 465.43* | 517.15* | 775.72* |
| fluid in tube | | | | | |
| Rotational speed of | 3-4 | 8 | 10** | 10** | 10** |
| peristatic pump | | | | | |

*The biomimetic microtubes were not fractured.
**The peristatic pump is MP-1000 type peristaltic pump produced by EYELA. The flow rate ranges from 10-1450 ml/h and is divided into 10 levels respectively corresponding to numbers 1-10.

TABLE 4
Tolerable pressure of various blood vessels

| blood vessels | vein | venule | capillary | arteriole | artery |
|---|---|---|---|---|---|
| Withstand | 5-10 | 8-12 | 12-25 | 25-40 | 40-100 |
| pressure (mmHg) | | | | | |

In summary, the walls of the biomimetic microtubes contain alginate cross-linked by divalent metal cations, so that the biomimetic microtube not only has the advantages of biocompatibility and non-toxicity, but also has sufficient pressure resistance and good permeability. Moreover, based on the test results of upper limit of the tolerable pressure of the biomimetic microtubes, it is known that the obtained biomimetic microtubes can easily withstand the pressure borne by the arteries.

In addition, the roughness, porosity, and hardness of the biomimetic microtubes can be changed by a variety of methods to simulate the condition of various blood vessels. For example, etching may be performed on the wall of the coaxial pipe to increase the roughness of the surface of the obtained biomimetic microtube. The roughness of the surface of the wall can be increased by adding a microphase substance, and the porosity of the wall can be increased even by a gaseous or liquid microphase substance. The biomimetic microtubes can be dehydrated by being immersed in an aqueous ethanol solution to increase the hardness of the biomimetic microtubes.

Therefore, the biomimetic microtubes obtained by the preparation method of biomimetic microtubes can be used to simulate blood vessels in various microenvironments in the body to perform various biomimetic experiments.

Although the disclosure has been disclosed by the above embodiments, the embodiments are not intended to limit the disclosure. It will be apparent to those skilled in the art that various modifications and variations can be made to the structure of the disclosure without departing from the scope or spirit of the disclosure. Therefore, the protecting range of the disclosure falls in the appended claims.

What is claimed is:

1. A method of preparing a biomimetic microtube, comprising:
providing a molding device, which comprises:
a coaxial pipe comprising an inner tube having an inner tube inlet and an inner tube outlet and an outer tube having an outer tube inlet and an outer tube outlet, wherein the outer tube sleeves outside the inner tube to form a two-layer coaxial pipe, and the outer tube outlet sleeves outside the inner tube outlet to form a coaxial outlet; and
a molding tank accommodating the coaxial outlet;
injecting a molding liquid containing a divalent metal cation into the molding tank, such that the coaxial outlet is positioned below a liquid surface of the molding liquid;
simultaneously introducing a gelatin solution from the inner tube inlet and an alginate solution containing a monovalent metal cation from the outer tube inlet, such that the gelatin solution and the alginate solution are simultaneously introduced into the molding liquid from the coaxial outlet to form a biomimetic microtube having a core solution and a wall surrounding the core solution.

2. The method of claim 1, wherein the monovalent metal cation is sodium ion, potassium ion or a combination thereof.

3. The method of claim 1, wherein the divalent metal cation is calcium ion, strontium ion or a combination thereof.

4. The method of claim 1, wherein the alginate solution comprises a plurality of microphase substances, and the microphase substances are a gas, a liquid, a solid or any combinations thereof.

5. The method of claim 4, further comprising heating the biomimetic microtube to remove the microphase substances from the biomimetic microtube to form pores in the wall of the biomimetic microtube when the microphase substances are the gas, the liquid or a combination thereof.

6. The method of claim 1, wherein an inner surface of the outer tube, an outer surface of the inner tube, or both are etched to increase roughness thereof.

7. The method of claim 1, further comprising immersing the biomimetic microtube in an aqueous solution of ethanol to dehydrate the biomimetic microtube.

8. A biomimetic microtube prepared by the method of claim 1.

9. The biomimetic microtube of claim 8, wherein the wall is a hydrogel from the alginate solution cross-linked by the divalent cations.

10. The biomimetic microtube of claim 8, wherein the core solution is from the gelatin solution and the alginate solution uncrosslinked by the divalent cations.

11. The biomimetic microtube of claim 8, wherein the monovalent metal cation is sodium ion, potassium ion or a combination thereof.

12. The biomimetic microtube of claim 8, wherein the divalent metal cation is calcium ion, strontium ion or a combination thereof.

13. The biomimetic microtube of claim 8, further comprising a plurality of microphase substances distributed in the wall, and the microphase substances are a gas, a liquid, a solid or any combinations thereof.

14. The biomimetic microtube of claim 13, wherein the wall has pores left after removing the microphase substances from the wall.

15. The biomimetic microtube of claim 13, wherein the wall has projections disposed thereon, and the projections are produced by the microphase substances.

16. The biomimetic microtube of claim 8, wherein the wall has a stripe pattern disposed thereon.

17. The biomimetic microtube of claim 8, wherein the wall is dehydrated.
